(19) United States
(12) Patent Application Publication
(10) Pub. No.: US 2021/0033440 A1
(43) Pub. Date: Feb. 4, 2021

(54) ULTRASONIC SYSTEM FOR DETECTING FLUID FLOW IN AN ENVIRONMENT

(71) Applicant: SUPERSONIC IMAGINE, AIX-EN-PROVENCE cedex 3 (FR)

(72) Inventors: David SAVÉRY, AIX-EN-PROVENCE (FR); Elsa GIRAUDAT, BULLION (FR)

(21) Appl. No.: 16/941,865

(22) Filed: Jul. 29, 2020

(30) Foreign Application Priority Data
Jul. 29, 2019 (FR) 1908627

(51) Int. Cl. G01F 1/66; A61B 8/06

(52) U.S. Cl. CPC G01F 1/663; A61B 8/06; G01F 1/667; G01F 1/662

(57) ABSTRACT

An ultrasonic system for detecting a fluid flow in an environment includes a probe configured for ultrasonic insonification of the environment and reception of an echo signal. The system also includes a control device configured to construct a series of images based on the signal received from the echoes. The images are filtered by a temporal high-pass filter. A local displacement of the flow between two successive images is determined by maximizing the similarity between blocks extracted from the two images.

### CROSS-REFERENCE TO RELATED APPLICATION

This application claims benefit of Serial No. 1908627, filed 29 Jul. 2019 in France and which application is incorporated herein by reference. To the extent appropriate, a claim of priority is made to the above disclosed application.

### BACKGROUND

An ultrasonic system can be used to determine the Doppler angle during a measurement of blood velocity (or of another liquid). The Doppler angle is the angle between the axis of the ultrasound beam and the velocity vector of the flowing blood. The Doppler method includes measuring an average frequency fDoppler from a plurality of pulses released at a constant rate PRF (Pulse Repetition Frequency), and using the Doppler formula to deduce therefrom the speed Vz projected on the axis of the beam. Speed Vz may be determined by the following equation:

Vz=c0*fDoppler/f0/2

where c0=speed of sound and f0=central frequency of the transmitted acoustic wave. In addition, the absolute speed Va=Vz/cos(Doppler angle) can be calculated. However, in the classical Doppler method, the scale of the Doppler spectrum is determined in a single region (i.e. the sampling volume) and therefore in a single beam.

As such, the user must manually identify and select this sampling volume in the ultrasound image in order to apply the Doppler method. Therefore, the angle must be manually positioned by the user in Pulsed Doppler (PD) mode, which does not allow automatic estimation of the direction of blood flow. For example, the user visually determines the direction of the vessel by inspecting the grayscale ultrasound image, then indicates via the user interface the direction of the estimated flow.

### SUMMARY

In one aspect, the technology relates to an ultrasonic system for detecting a fluid flow in an environment, the system including: a probe configured for ultrasonic insonification of the environment and reception of an echo signal, a control device configured to: build a sequence of images based on the echo signal, filter the images by a temporal high-pass filter, and determine a local displacement of the fluid flow between two successive images of the sequence of images by maximizing the similarity between blocks extracted from the two successive images. In an example, the blocks include at least one first reference block in a first of the two successive images and at least one second comparison block having the same size in a second of the two successive images, and in which the similarity is maximized by optimizing a position of the second block. In another example, the control device is configured to: calculate at least one of an average angle and an average speed of the fluid based on the determined local displacement, and wherein the average angle being defined with respect to a reference axis of the probe. In yet another example, the high-pass filter includes a high-pass infinite impulse response filter, or a matrix filter of orthogonal projection on a predefined subspace. In still another example, the determination of the local displacement of the fluid flow includes calculating in each pixel an angle of the local displacement.

In another example of the above aspect, the control device is configured to detect one or more channels in the environment carrying the fluid by segmenting homogeneous regions in the sequence of images, and in which the control device is configured to calculate at least one of the average angle and the average speed of the fluid for each channel of the one or more channels. In an example, the segmentation is based on a predefined decision rule on the amplitude and/or average frequency of the signals, which construct the sequence of high-pass filtered images. In another example, the determination of the local displacement of the flow includes applying a block-matching algorithm to locate similar blocks between two successive images. In yet another example, the block-matching algorithm is configured to maximize the spatial intercorrelation between two windows of two successive filtered images. In still another example, the block-matching algorithm uses an increasing function of an envelope of a plurality of Doppler signals filtered between two successive images to determine a local displacement in a set of pixels.

In another example of the above aspect, the block-matching algorithm is configured to use an envelope of a plurality of Doppler signals filtered between two successive images to determine a local vector displacement in a set of pixels. In an example, the block-matching algorithm includes at least one of the following algorithms: temporal and spatial averaging to estimate a two-dimensional spatial intercorrelation function and maximizing the two-dimensional spatial intercorrelation function, and minimization of a sum of absolute differences function. In another example, the control device is configured to calculate the circular variance of an angle of fluid flow based on the local displacement determined over a set of pixels. In yet another example, the set of pixels is a set of pixels in a channel. In still another example, the probe is configured for at least one of: ultra-fast insonification with a rate of at least 500 pulses per second, and insonification at different angles, and insonification with a succession of pulses of at least one of ultrasonic plane waves firing from variable angles and ultrasonic cylindrical waves firing from variable sources, and the control device is configured to construct a series of demodulated baseband images for a repetition rate of pulses of at least 500 Hz.

In another aspect, the technology relates to a method of detecting a fluid flow in an environment, the method including: ultrasonically insonifying the environment, receiving an echo signal, constructing a series of images based on the echo signal, filtering the series of images by a temporal high-pass filter, and determining a local displacement of the fluid flow between two successive images of the series of images by maximizing the similarity between blocks extracted from the two successive images. In an example, ultrasonically insonifying the environment includes emitting an ultrasound signal with a rate of at least about 500 pulses per second into the environment. In another example, the determination of the local displacement of the flow includes applying a block-matching algorithm to locate similar blocks between the two successive images.

In another aspect, the technology relates to a non-transitory computer-readable storage medium including computer executable instructions that, when executed by a processor, perform operations, including: receiving information associated with an echo signal in response to ultrasonically insonifying an environment; constructing a series of images based on the echo signal; filtering the series of images by a temporal high-pass filter; and determining a local displacement of the fluid flow between two successive images of the series of images by maximizing the similarity between blocks extracted from the two successive images. In an example, the determination of the local displacement of the flow includes applying a block-matching algorithm to locate similar blocks between the two successive images.

### BRIEF DESCRIPTION OF DRAWINGS

FIG. 1 is a schematic view of an architecture of an ultrasonic system according to one example of the technology.

FIGS. 2A-2C depict a schematic view of a method for processing constructed ultrasonic data to determine a fluid flow in an environment according to one example of the technology.

FIG. 3 schematically illustrates an example of a determined block in a vessel at a time t.

FIG. 4 schematically illustrates the calculation of the local displacement of the block of FIG. 3 at a time t+1/PRF.

FIG. 5 schematically illustrates the calculation of the local speed resulting from the local displacement of the block of FIG. 4.

FIG. 6 schematically illustrates an example of a calculation of local speeds and angles.

### DETAILED DESCRIPTION

The disclosed technologies generally relate to ultrasound and ultrasonic systems, for example for medical ultrasound investigation. The technology relates further to ultrasonic systems for detecting a fluid flow in an environment, for example to automatically determine the direction of blood flow.

The technologies described herein allow automatic estimation of the direction of flow of blood in one or more vessels in an ultrasound image.

To this end, the technology provides an ultrasonic system for detecting a fluid flow in an environment. The environment may include a probe configured for ultrasonic insonification of the environment and reception of the echo signal. An associated control device is configured to build, rebuild, or construct a series of images based on the signal received from the echoes. The control device may filter the images utilizing a temporal high-pass filter and determine a local displacement of the flow between two successive images by maximizing the similarity between blocks extracted from the two images.

Therefore, thanks to such a control device, the direction of flow of blood in one or more vessels in an ultrasound image can be estimated automatically. In particular, the method implemented by the system makes it possible to display speed spectrums whose Doppler angle is, for example, determined automatically.

Furthermore, the system according to examples of the present disclosure makes it possible to estimate the Doppler spectrums at any point in the environment examined by the system. This allows the direction of the blood flow in several vessels to be estimated simultaneously. In comparison, in conventional Doppler as described above, the scale of the Doppler spectrum is determined in a single region (the sampling volume) and the user must identify and manually select this volume in the ultrasound image.

In particular, the system allows the evaluation of angles on a plurality of elements in the image (for example blood vessels).

Thus, it is possible to assess the uncertainty of estimated angles, which is useful for assessing the accuracy and/or variance of the measure.

Advantageously, the disclosed method makes it possible to obtain a spatial resolution equivalent to the coherent image formed by a large aperture and a set of angles of pulses which is therefore better than that obtained by using a sub-aperture for reception.

In addition, compared to known methods known as “speckle tracking” on B-mode data, the system described herein is advantageously more sensitive to blood flow. Doppler data can for example be filtered by a selective wall filter of the blood flow and suppressor of stationary tissues,

In addition, the method made available by the system is better in measuring absolute speed than the other known Doppler methods, with multiple beams, in particular when the Doppler angle is large (and therefore the projection error is large), since the absolute speed value is all the greater the larger the Doppler angle is, the speed of the amplitude being proportional to the inverse of the cosine of the Doppler angle. For example, when the inspected vessel is horizontal on an ultrasound image, and when the Doppler beams are almost vertical (for reasons of directivity of the transducer elements, and therefore of signal-to-noise ratio), then the Doppler angle(s) are large, and the error on the absolute speed due to the uncertainty of the angle is high.

The system makes it possible to calculate new clinical diagnostic indices, for example of the liver (e.g. assess the isotropy/anisotropy of liver perfusion by characterizing a plurality of angles on several vessels).

The images can be filtered by a temporal high-pass filter for example, making it possible to eliminate the signal coming from the stationary environment.

The similarity between two blocks can be expressed for example by the spatial intercorrelation between the two blocks.

The environment can be stationary or quasi-stationary. It can be a tissue, for example, an organ (for example the liver) or a muscle.

The blocks may include at least one first reference block in the first of the two images and at least one second comparison block having the same size in the second of the two images. For example, the block-matching algorithm can be applied to optimally determine which the two blocks can be. Indeed, this algorithm makes it possible to locate similar blocks between two images.

The similarity can be maximized by optimizing (changing) the position of the second block, for example to find the first block in the second image.

The control device can be configured to calculate an average angle and/or an average speed of the fluid based on the determined local displacement.

The angle can be defined for example with respect to a reference axis of the probe.

The high-pass filter can include a high-pass infinite impulse response filter, or a matrix filter of orthogonal projection on a predefined subspace.

Determining the local displacement of the flow can include calculating the angle of the local displacement in each pixel.

Determining the local displacement of the flow can include a segmentation of the images into homogeneous regions. Each of the regions can correspond to a channel, for example a vessel, carrying the fluid.

Consequently, the control device can be configured to detect one or more channels in the environment carrying the fluid by segmenting homogeneous regions in the images.

The control device can be configured to calculate an average angle and/or an average speed of the fluid for each of the channels.

The segmentation can be based on a predefined decision rule on the amplitude and/or the average frequency of the high-pass filtered signals.

Determining the local displacement of the flow can include the use of a block-matching algorithm, for example by locating similar blocks between two successive images. The block-matching algorithm can use, for example, a predefined block size and a predefined pixel progression step.

The block-matching algorithm can be configured to maximize the spatial intercorrelation between two windows of two successive filtered images

The block-matching algorithm can use an increasing function of the envelope of the Doppler signals filtered between two successive images to determine a local displacement in a set of pixels (for example a set of pixels in a channel).

The block-matching algorithm can be configured to use the envelope of the filtered signals between two successive images to determine a local vector displacement in a set of pixels (for example a set of pixels in a channel).

In an example, the block-matching algorithm may utilize temporal and/or spatial averaging to estimate the 2D spatial intercorrelation function then maximize it. In another example, the block-matching algorithm may utilize minimization of the sum of absolute differences function.

The control device can be configured to calculate the circular variance of the angle of flow based on the local displacement determined on a set of pixels (for example a set of pixels in a channel).

The pixel set can be a set of pixels in a channel or pixels that form the channel on the images.

The probe can be configured for insonification at rates of at least about 500 pulses per second, at least about 1000 pulses per second, at least about 1500 pulses per second, at least about 2000 pulses per second, at least about 2500 pulses per second, or at least about 3000 pulses per second. Other rates are contemplated, and may include rates up to at least about 9000 pulses per second and at least about 10,000 pulses per second. In certain examples, insonification at certain rates may be referred to as ultra-fast insonification.

The probe can be configured for insonification at different angles.

The probe can be configured for insonification with a succession of ultrasonic plane wave firing from variable angles or ultrasonic cylindrical wave firing from variable source points.

The control device can be configured to construct a series of demodulated baseband images for a typical firing repetition rate of for such as those noted above, for example.

The probe may include an array of ultrasonic transducers and/or an array of ultrasonic transducers.

The technology also provides an ultrasonic method for detecting a fluid flow in a environment. The method includes

insonifying the environment and receiving an echo signal;

building a series of images based on the signal received from the echoes;

filtering the images by a temporal high-pass filter; and determining a local displacement of the flow between two successive images by maximizing the similarity between blocks extracted from the two images.

Other details and advantages of the presented technology will be apparent on reading the description that follows, given solely by way of non-limiting example, and made with reference to the accompanying drawings.

### Example

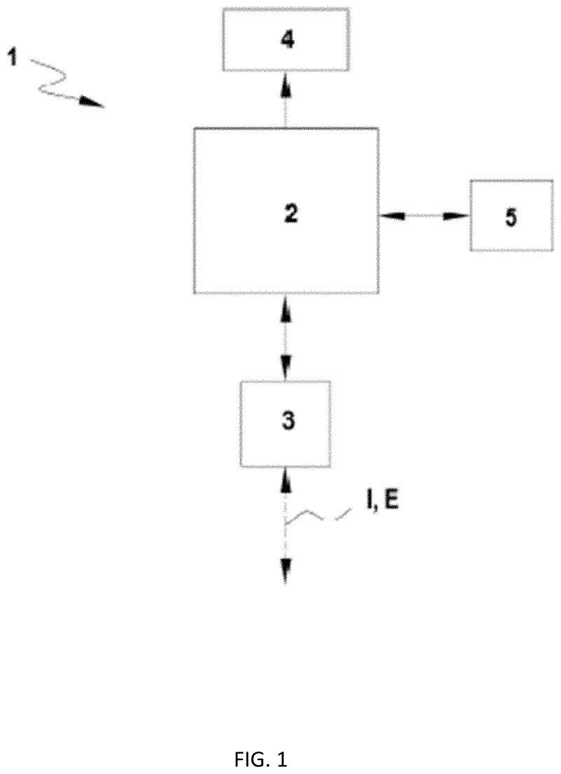

FIG. 1 is a schematic view of an architecture of an ultrasonic system 1 according to one example of the present technology. The system 1 may be an electronic display system and it may for example be configured for determining the Doppler angle during a medical ultrasound investigation. For example, it can automatically estimate the direction of blood flow.

The system 1 comprises a probe 3 configured for ultrasonic insonification I of the environment and reception of an echo signal E. The probe can include an array of ultrasound transducers and/or a matrix of ultrasound transducers.

In addition, the system includes a control device 2 coupled with the probe 3 and capable of capturing a series of images of an environment using the probe 3. The device 2 in an example of use, constructs (or reconstructs) the sequence of images based on the signal received from the echoes, filters the images by a temporal high-pass filter, and determines a local displacement of the flow between two successive images by maximizing the similarity between blocks extracted from the two images. Advantageously, the device can therefore automatically estimate the direction of flow of the liquid (for example blood) in one or more vessels in one of the ultrasound images obtained using the probe. In particular, the device according to the disclosure makes it possible to display speed spectrums whose Doppler angle is for example determined automatically.

Furthermore, the system may include a first screen 4 and optionally another second screen 5 which may be a touchscreen. Screen 5 can be a one-touch or multi-touch screen. At least one of the screens can display the speed spectrums. In examples, the control device 2 and screens 4, 5 may be incorporated into a single device. In other examples, screens 4, 5 may be computer or other screens disposed remotely from the control device 2.

The disclosed system is capable of performing the following operations:

1. Insonification (in some examples, ultra-fast) of the environment by ultrasonic plane waves at different angles (for example two, three, four, or more angles, insonification at a rate such as described herein) or by cylindrical ultrasonic waves at different source points and reception by the probe 3. Waves having other shapes are also contemplated.

The following operations may be performed by the control device 2:

2. Construction of a sequence of IQ images for a typical PRF rate of for example 3000 Hz, or other rates as indicated elsewhere herein;

3. Filtering by wall filter to eliminate the signal coming from stationary tissues;

4. Block matching using the envelope of the Doppler signals filtered between two successive instants to determine a local displacement in a set of pixels (which are the centers of the blocks). The block-matching method can include maximizing the spatial intercorrelation between two successive filtered Doppler images.

5. (Optional Operation) Calculation in each pixel of the angle of the local displacement, and

6. (Optional Operation) Calculation on each vessel of an average angle (spatial average restricted to one vessel region), an average speed, and/or an average speed vector.

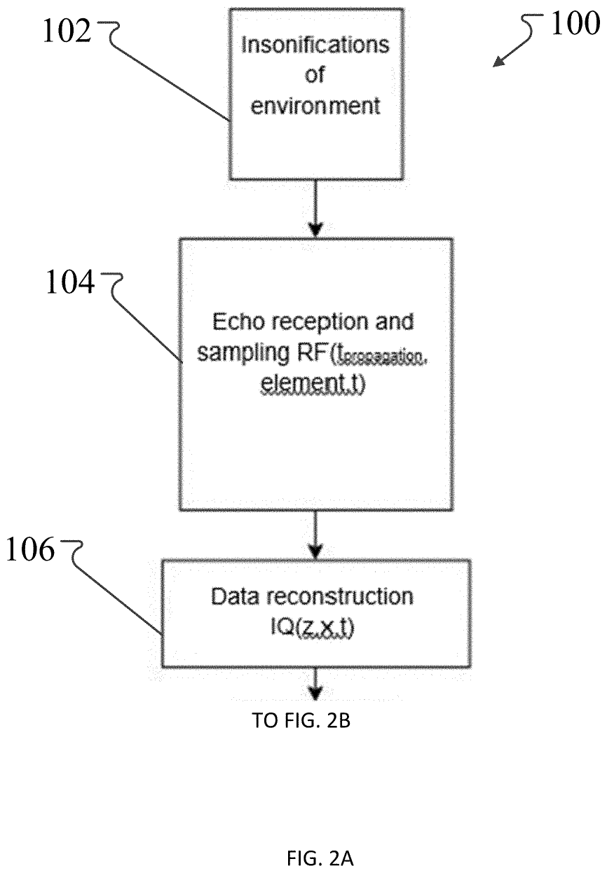

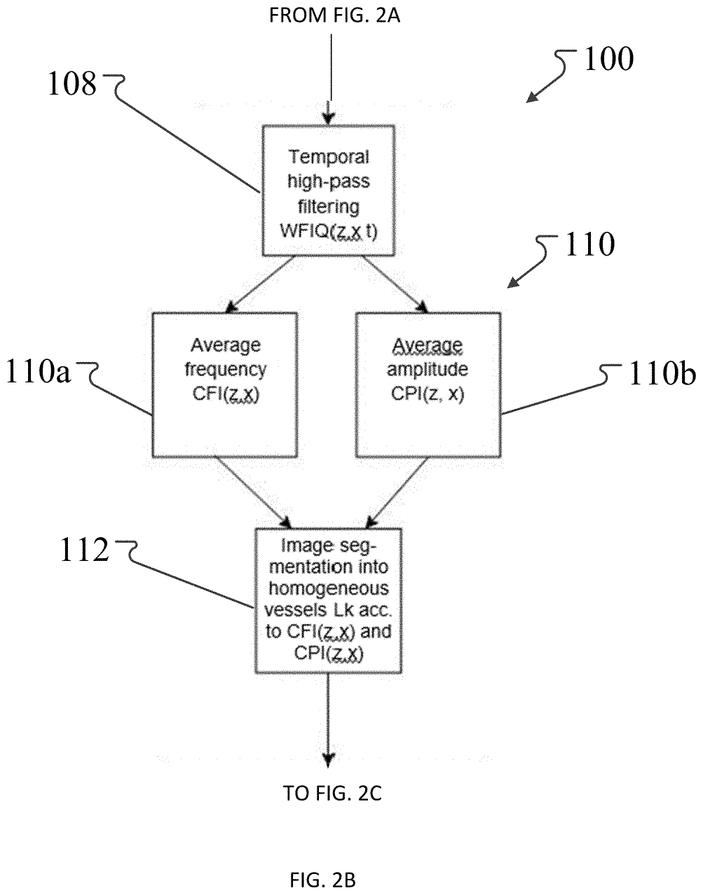

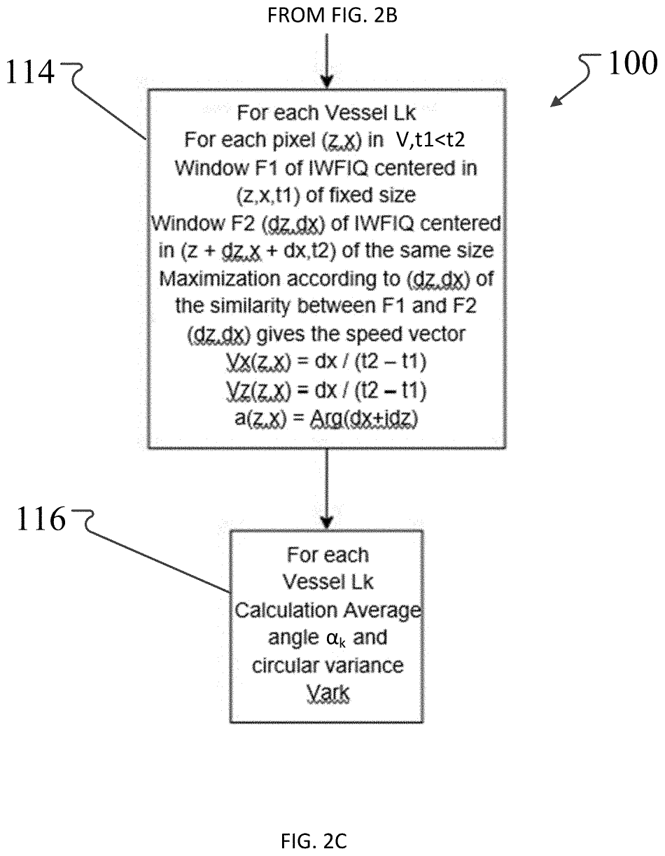

Operations 2 to 6 will be described in more detail in the context of FIGS. 2A-2C.

FIGS. 2A-2C are a schematic view of a method 100 of processing constructed ultrasonic data to determine a fluid flow in an environment according to one example of the technology. The method 100 may include one or more of the following operations:

FIG. 2A depicts obtaining a series of images constructed on the basis of the echo signal. More specifically, the method 100 includes, at operation 102, periodic insonification of the environment at a typical PRF rate of for example 0.5 to 10 kHz (as noted elsewhere herein) by a succession of EL pulses of ultrasonic plane waves from variable angles or of ultrasonic cylindrical waves with variable apices (center of curvature of wave fronts) emitted by an array or matrix of ultrasonic transducers. Operation 104 includes reception and sampling of waves backscattered by the environment (amplification and associated filtering). Operation 106 includes construction of a plurality of demodulated images, coherent summation on the angles or on the apices, to obtain a series of complex images IQ(z,x,t), where the slow time is sampled at the frequency PRF and where the number of demodulated images obtained is designated by EL, Ensemble Length or “packet size”.

FIG. 2B depicts further operation of the method 100, including operation 108, which includes filtering the images by a high-pass filter (for example a temporal “wall filter”). operation 108 may further include high-pass (“wall”) filtering of the image sequence for the elimination of fixed or quasi-fixed echoes to obtain a new sequence WFIQ(z,x,t). This filtering can take the form of a linear operation characterized by an EL*EL pixel-dependent transformation matrix of the form:

[WFIQ(z,x,1) . . . WFIQ(z,x,EL)]^{T}=M(z,x)[IQ(z,x,1) . . . IQ(z,x,EL)]^{T} (eq. 1)

M(z,x) can be obtained from the impulse response of an invariant linear filter with finite or infinite impulse response, or can be defined as a projection matrix on a subspace of C^{EL} (polynomials of degrees above a fixed order, trigonometric polynomials, etc.), this subspace characterizing the ultrasonic echoes of the flowing blood. M(z,x) can be variable with the pixel (z,x) or be constant in the image.

In operation 110, an average velocity map and an average amplitude map are calculated. In more detail, operation 110 includes operation 110a, extraction of the average frequency CFI(z,x) (color flow image); and operation 110b, extraction of the average amplitude CPI(z,x) (color power image) in each pixel of coordinates (z,x) from the vector [WFIQ(z,x,1) . . . WFIQ (z,x,EL)]. This average frequency and this average amplitude can be calculated for example from the following relationships (* denotes the complex conjugate, arg the argument for [−pi, pi] of a complex number):

[Math. 1]

CPI=[1/(EL−1)>t=1 . . . E_{L}WFI_{Q(z,x,t)*}WFIQ_{(z,x,t})]^{0.5} (eq. 2)

CPI=PRF*arg(Σt=1 . . . E_{L}WFI_{Q(z,x,t)*}WFI_{Q(z,x,t+1)}/2/pi (eq. 3)

The two CFI and CPI maps thus obtained are then used to make it possible to segment and label the vessels. The larger I CFI (z, x) I and CPI (z, x) are, the greater is the probability that (z, x) is in a vessel.

In operation 112, a vessel (e.g. a blood vessel) is located in the average velocity map. This operation may include segmentation into homogeneous regions Lk, k=1 . . . Nk (which can be reduced to one pixel) according to the values of CPI and CFI, with each of the regions corresponding to a vessel. A segmentation method that can be used is to divide the CPI image into related components including the CPI value (z, x) and |CFI (z, x)| are above predefined thresholds.

The method 100 continues in FIG. 2C where, at operation 114, a local displacement is determined for each pixel in the vessel, for example using the block-matching algorithm. Operation 114 may include, for example, for each pixel (z,x) of each homogeneous region Lk, and for a time pair t1<t2, the calculation of the local speed vector v=(dz,dx)*1/(t1−t2) is performed by maximizing the correlation index C(dz,dx) defined by:

$$\begin{matrix} Correlation Index ( dz , dx ) = \frac{{A}_{12} ( dz , dx )}{\sqrt{{A}_{11} * {A}_{22}}} & [ Math . 2 ] \\ {A}_{11} = {\sum }_{window} {\vert WFIQ ( z , x , t 1 ) \vert}^{⋀} 2 & \\ {A}_{22} = {\sum }_{translated window} {\vert {WFIQ}^{*} ( z , x , t 2 ) \vert}^{⋀} 2 & \\ {A}_{12} ( dz , dx ) = {\sum }_{window} \vert {WFIQ}^{*} ( z , x , t 1 ) \vert \vert WFIQ ( z + dz , x + dx , t 2 ) \vert & \end{matrix}$$

where “window” indicates a window centered in (z,x) whose size is predefined in (z,x).

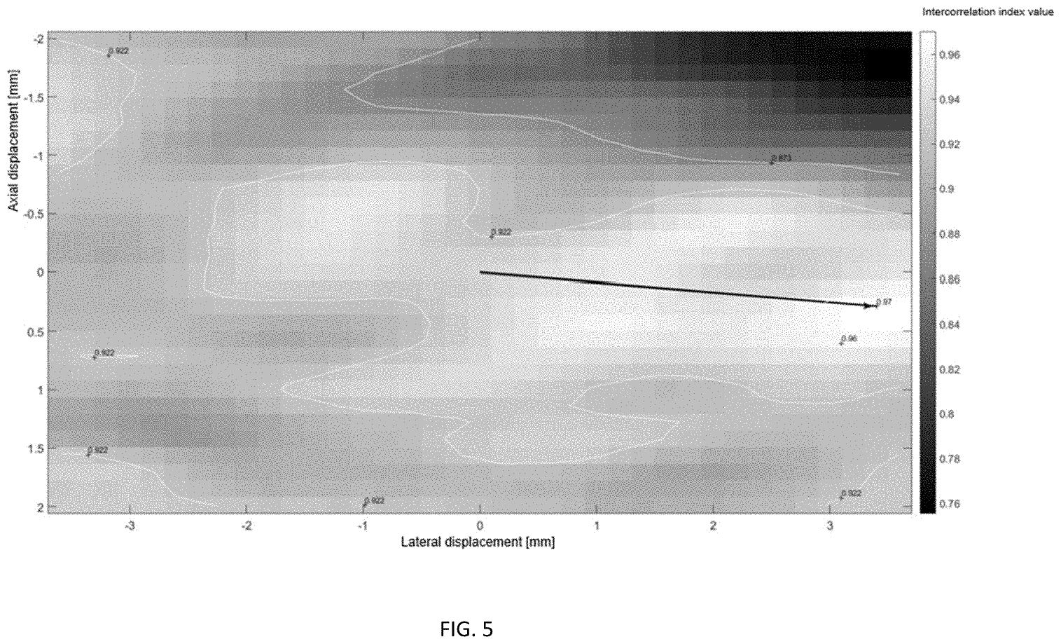

As shown in the example in FIG. 5, the correlation index can be estimated more robustly by averaging its values both spatially and over time t1 and t2, considering pairs t1 and t2 such that t2−t1 is a constant.

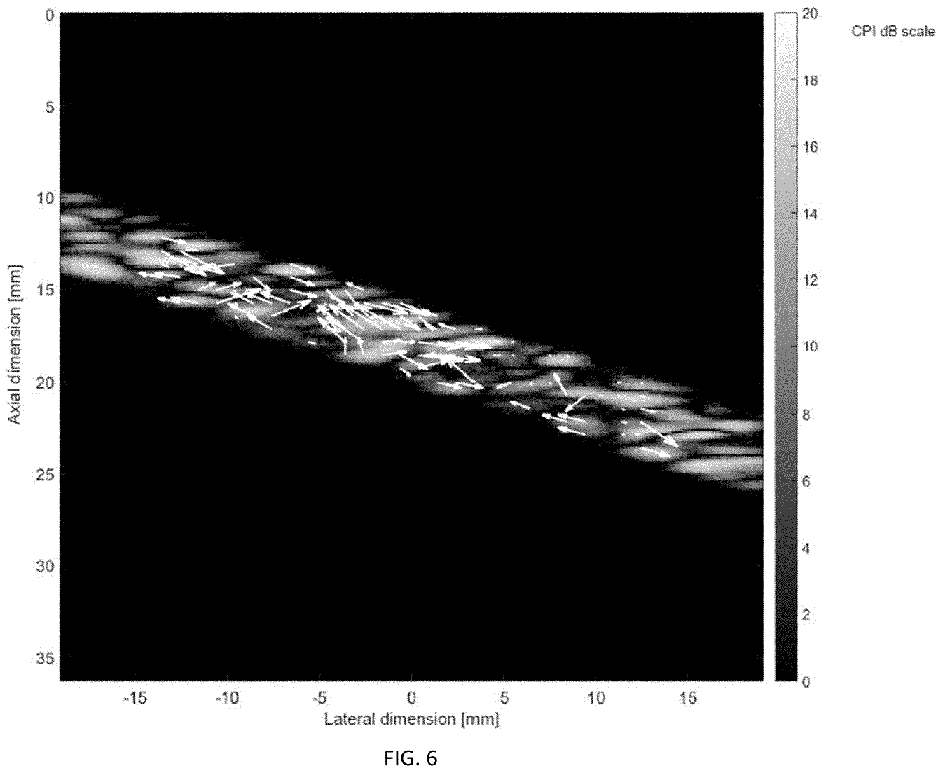

As shown in the example in FIG. 6, if the maximized correlation index does not exceed a predefined threshold, then the estimate of the local displacement (i.e. each arrow indicated in FIG. 6) can be of poor quality and local speed [Vz(z,x) Vx(z,x)] may not be taken into account in the following for the calculation of certain statistics for example. Therefore, the predefined threshold advantageously improves the estimation of the average speed and angle by eliminating outliers.

In optional operation 116, for each homogeneous region Lₖ, an average angle calculation αₖ can then be extracted, as well as a circular variance varₖ. These values can for example be calculated using the following formulas:

e(z,x)=[Vx(z,x)+i Vz(z,x)]/|Vx(z,x)+i Vz(z,x)| (eq. 4)

αₖ=arg(<e(z,x)>ₖ) (eq. 5)

varₖ=1−|<e(z,x)>ₖ|, (eq. 6)

where the averaging < >ₖ is located on the homogeneous region Lₖ.

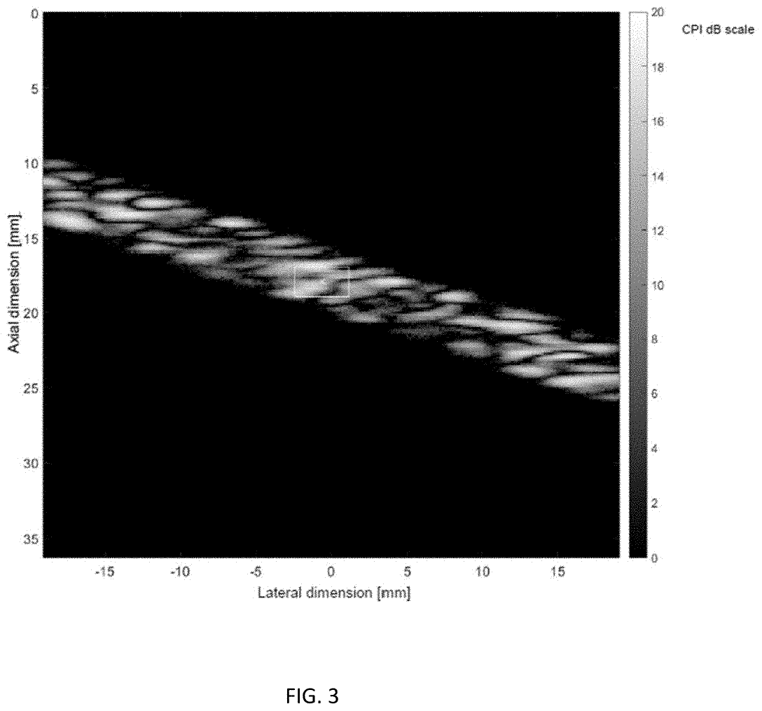

In this context, an example is illustrated in FIGS. 3 to 6.

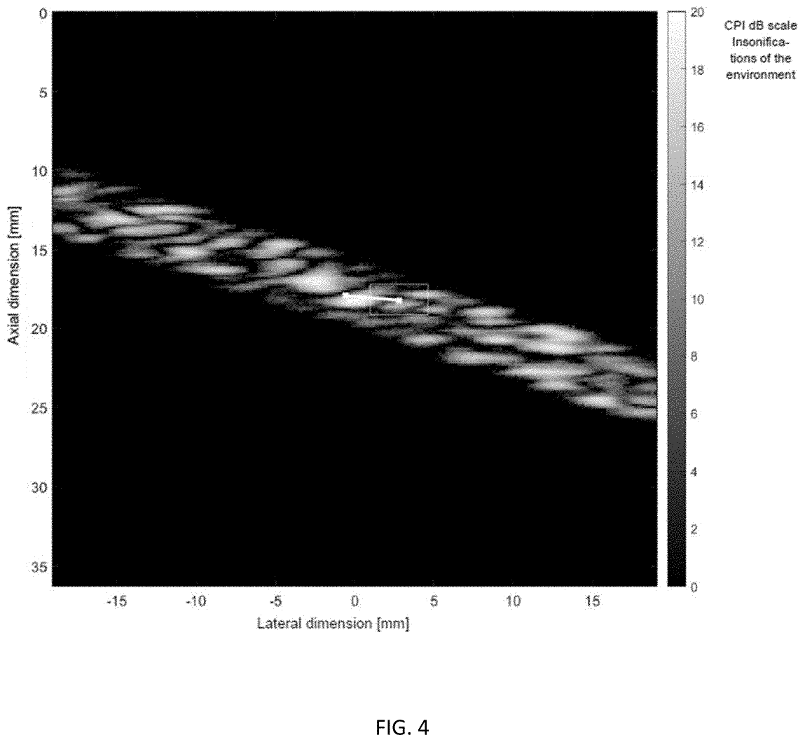

FIGS. 3 and 4 schematically illustrate respectively an example of a determined block in a vessel at a time t, and the calculation of the local displacement of the block at a time t+1/PRF. In particular, FIG. 3 illustrates an image of the amplitude of a Doppler image of an inclined vessel at a point in time t1. The white rectangle represents a window F1 (that is to say a block, for example determined by the block-matching algorithm) of chosen size. FIG. 4 illustrates the image of the amplitude of a Doppler image of an inclined vessel for example at a point in time t2=t1+5.7 ms (or another predefined value). The white rectangle represents the window F2(dz,dx) which maximizes the spatial intercorrelation between F1 and an offset window. The displacement evaluated at the center of F1 corresponds to the white vector.

FIG. 5 schematically illustrates the calculation of the local speed resulting from the local displacement of the block of FIG. 4, that is to say, the intercorrelation function between the two windows illustrated in FIGS. 3 and 4. In particular, FIG. 5 illustrates the image of the spatial intercorrelation (and its level lines) between F1 and F2(dz,dx). It is maximized by for example dx=3.4 mm and dz=0.3 mm for an index of for example 0.97. It is the estimator of the displacement vector between t1 and t2 at the center of F1.

FIG. 6 schematically illustrates an example of a calculation of local speeds and angles. In particular, FIG. 6 illustrates an image of the velocity vectors calculated on a pixel grid of an inclined vessel.

1. An ultrasonic system for detecting a fluid flow in an environment, comprising:
a probe configured for ultrasonic insonification of the environment and reception of an echo signal,
a control device configured to:
build a sequence of images based on the echo signal,
filter the images by a temporal high-pass filter, and
determine a local displacement of the fluid flow between two successive images of the sequence of images by maximizing the similarity between blocks extracted from the two successive images.

2. The ultrasonic system according to claim 1, in which the blocks comprise at least one first reference block in a first of the two successive images and at least one second comparison block having the same size in a second of the two successive images, and in which the similarity is maximized by optimizing a position of the second block.

3. The ultrasonic system according to claim 1, in which the control device is configured to:
calculate at least one of an average angle and an average speed of the fluid based on the determined local displacement, and wherein the average angle being defined with respect to a reference axis of the probe.

4. The ultrasonic system of claim 1, wherein the high-pass filter comprises a high-pass infinite impulse response filter, or a matrix filter of orthogonal projection on a predefined subspace.

5. The ultrasonic system of claim 1, in which the determination of the local displacement of the fluid flow comprises calculating in each pixel an angle of the local displacement.

6. The ultrasonic system of claim 1, in which the control device is configured to detect one or more channels in the environment carrying the fluid by segmenting homogeneous regions in the sequence of images, and in which the control device is configured to calculate at least one of the average angle and the average speed of the fluid for each channel of the one or more channels.

7. The ultrasonic system according to claim 6, wherein the segmentation is based on a predefined decision rule on the amplitude and/or average frequency of the signals, which construct the sequence of high-pass filtered images.

8. The ultrasonic system of claim 1, in which the determination of the local displacement of the flow comprises applying a block-matching algorithm to locate similar blocks between two successive images.

9. The ultrasonic system of claim 8, in which the block-matching algorithm is configured to maximize the spatial intercorrelation between two windows of two successive filtered images.

10. The ultrasonic system of claim 8, in which the block-matching algorithm uses an increasing function of an envelope of a plurality of Doppler signals filtered between two successive images to determine a local displacement in a set of pixels.

11. The ultrasonic system of claim 8, wherein the block-matching algorithm is configured to use an envelope of a plurality of Doppler signals filtered between two successive images to determine a local vector displacement in a set of pixels.

12. The ultrasonic system of claim 8, in which the block-matching algorithm comprises at least one of the following algorithms:
temporal and spatial averaging to estimate a two-dimensional spatial intercorrelation function and maximizing the two-dimensional spatial intercorrelation function, and
minimization of a sum of absolute differences function.

13. The ultrasonic system of claim 1, in which the control device is configured to calculate the circular variance of an angle of fluid flow based on the local displacement determined over a set of pixels.

14. The ultrasonic system of claim 10, wherein the set of pixels is a set of pixels in a channel.

15. The ultrasonic system of claim 1, in which the probe is configured for at least one of:
ultra-fast insonification with a rate of at least 500 pulses per second, and insonification at different angles, and
insonification with a succession of pulses of at least one of ultrasonic plane waves firing from variable angles and ultrasonic cylindrical waves firing from variable sources, and
the control device is configured to construct a series of demodulated baseband images for a repetition rate of pulses of at least 500 Hz.

16. A method for detecting a fluid flow in an environment, the method comprising:
ultrasonically insonifying the environment,
receiving an echo signal,
constructing a series of images based on the echo signal,
filtering the series of images by a temporal high-pass filter, and
determining a local displacement of the fluid flow between two successive images of the series of images by maximizing the similarity between blocks extracted from the two successive images.

17. The method of claim 16, wherein ultrasonically insonifying the environment comprises emitting an ultrasound signal with a rate of at least about 500 pulses per second into the environment.

18. The method of claim 17, wherein the determination of the local displacement of the flow comprises applying a block-matching algorithm to locate similar blocks between the two successive images.

19. A non-transitory computer-readable storage medium comprising computer executable instructions that, when executed by a processor, perform operations, comprising:
receiving information associated with an echo signal in response to ultrasonically insonifying an environment;
constructing a series of images based on the echo signal;
filtering the series of images by a temporal high-pass filter; and
determining a local displacement of the fluid flow between two successive images of the series of images by maximizing the similarity between blocks extracted from the two successive images.

20. The method of claim 19, wherein the determination of the local displacement of the flow comprises applying a block-matching algorithm to locate similar blocks between the two successive images.
